(19) United States
(12) Patent Application Publication
(10) Pub. No.: US 2008/0015197 A1
(43) Pub. Date: Jan. 17, 2008

(54) Process for the preparatrion of zopiclone

(76) Inventors: Alex Mainfeld, Kfar-Saba (IL); Marioara Mendelovici, Rehovot (IL)

Correspondence Address: KENYON & KENYON LLP, ONE BROADWAY, NEW YORK, NY, 10004, US

(21) Appl. No.: 11/823,345

(22) Filed: Jun. 26, 2007

Related U.S. Application Data
(60) Provisional application No. 60/816,328, filed on Jun. 26, 2006.

(51) Int. Cl. A61K 31/4985; A61P 25/20; C07D 487/04

(52) U.S. Cl. 514/249; 544/350

(57) ABSTRACT

Provided is a process for the preparation of zopiclone, an intermediate in the synthesis of eszopiclone.

### CROSS REFERENCE TO RELATED APPLICATIONS

The present application claims the benefit of the following U.S. Provisional Patent Application No. 60/816,328, filed Jun. 26, 2006. The contents of the application are incorporated herein by reference.

### FIELD OF THE INVENTION

The present invention is directed to the preparation of zopiclone, an intermediate in the synthesis of eszopiclone.

### BACKGROUND OF THE INVENTION

Zopiclone, a non-benzodiazepine sedative-hypnotic useful for treating insomnia, is a racemate having a chemical name of 4-methyl-1-piperazinecarboxylic acid 6-(5-chloro-2-pyridinyl)-6,7-dihydro-7-oxo-5H-pyrrolo[3,4-b]pyrazin-5-yl ester, (±)-6-(5-chloro-2-pyridinyl)-6,7-dihydro-7-oxo-5H-pyrrolo[3,4-b]pyrazin-5-yl-4-methylpiperazine-1-carboxylate, or 6-(5-chloropyrid-2-yl)-5-(4-methylpiperazin-1-yl)carbonyloxy-7-oxo-6,7-dihydro-5H-pyrrolo[3,4-b]pyrazine, represented with formula I below.

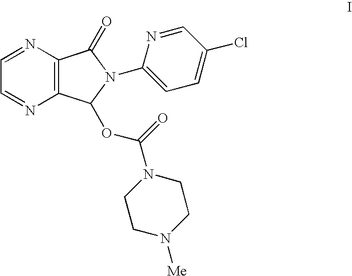

Eszopiclone is the S-enantiomer of zopiclone and is more active and less toxic than the racemic zopiclone according to U.S. Pat. No. 6,444,673 B1. Eszopiclone has a chemical name of (+)-6-(5-chloro-2-pyridinyl)-7(S)-(4-methylpiperazin-1-yl-carbonyloxy)-6,7-dihydro-5H-pyrrolo[3,4-b]pyrazine-5-one and is represented with formula II below.

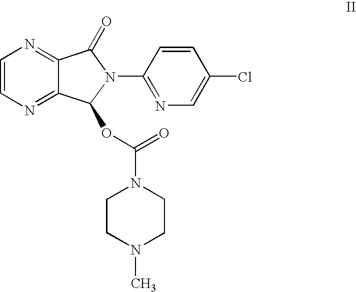

U.S. Pat. No. 3,862,149 discloses a process for preparing zopiclone as shown in Scheme 1 below. The process comprises combining 6-(5-chloro-2-pyridinyl]-6,7-dihydro-7-hydroxy-5H-pyrrolo[3,4-b]pyrazine-5-one (7-OH-Py) 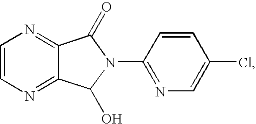
which is one of the intermediates in the synthesis, with chloro-carbonyl-4-methyl-piperazine (CMP) 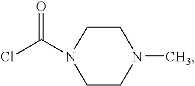
in the presence of N,N-dimethyl-formamide as a solvent and NaH as a base. After chromatographic purification and recrystallization from a mixture of acetonitrile and diisopropyl ether, zopiclone is obtained with a yield of 47%.

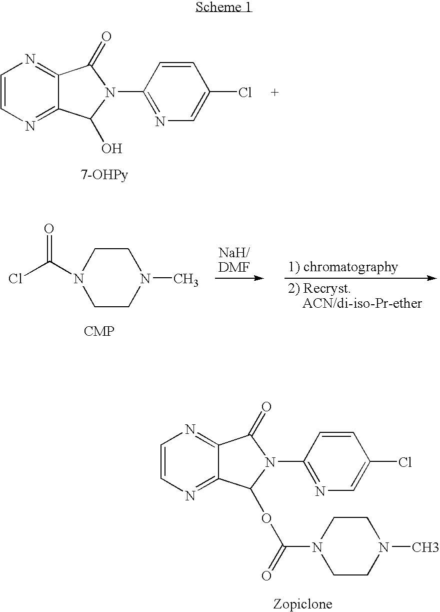

As apparent, the process described in U.S. Pat. No. 3,862,149 is reported to give a yield of zopiclone that is relatively low and, thus, the crude reaction product needs purification by chromatography and crystallization. U.S. Pat. No. 3,862,149 also describes the use of NaH as a base, which is a hazardous chemical for industrial processes. NaH also activates the substrate 7-OH-Py, and as a result there is a need for at least two equivalents of this hazardous reagent.

Comptes Rendue Acad. Sci. Paris, 287, Serie C, 1978, pp. 377 discloses the preparation of zopiclone by combining 7-OH-Py with CMP hydrochloride salt in methylene chloride in the presence of pyridine as a catalyst and triethylamine. This reference does not provide a detailed experimental example. Usually when pyridine is used, it is used in excess and acts as a reaction solvent. The use of pyridine is not recommended for pharmaceuticals formulations. The limit of Pyridine in pharmaceutical active ingredients is 200 ppm.

US2007/0054914, which published after the priority date of the present application, discloses preparation of zopiclone by using the reagents 7-OH— and CMP HCl, and either potassium carbonate and t-butyl ammonium bromide, or N,N-dimethylformamide and sodium hydride.

Thus, there is a need for an improved process for preparing zopiclone with improved properties.

### SUMMARY OF THE INVENTION

In one of the embodiments, the present invention provides a process for preparing zopiclone by combining 6-(5-chloro-2-pyridinyl]-6,7-dihydro-7-hydroxy-5H-pyrrolo[3,4-b]pyrazine-5-one (7-OH-Py) having the formula: 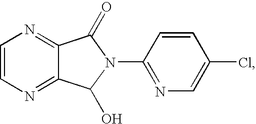
With chloro-carbonyl-4-methyl-piperazine (CMP) as a free base or an acid addition salt having the formula: 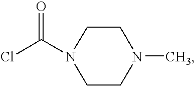
4-N,N-dimethylamino-Pyridine (DMAP) catalyst and a base in the presence of a polar solvent.

In another embodiment, the present invention provides a process for preparing eszopiclone, comprising preparing zopiclone by the process of the present invention, and converting it to eszopiclone.

In yet another embodiment, the present invention provides a pharmaceutical composition comprising eszopiclone made by the process of the present invention and at least one pharmaceutically acceptable excipient.

In one of the embodiments, the present invention provides a process for preparing a pharmaceutical formulation comprising combining eszopiclone made by the process of the present invention, with at least one pharmaceutically acceptable excipient.

In another embodiment, the present invention provides the use of eszopiclone made by the process of the present invention for the manufacture of a pharmaceutical composition.

### DETAILED DESCRIPTION OF THE INVENTION

As used herein, the term “7-OH-Py” refers to 6-(5-chloro-2-pyridinyl]-6,7-dihydro-7-hydroxy-5H-pyrrolo[3,4-b]pyrazine-5-one. The starting material 7-OH-Py may be prepared according to the process described in U.S. Pat. No. 3,862,149.

As used herein, the term “CMP” refers to chloro-carbonyl-4-methyl-piperazine as a free base or as an acid addition salt.

As used herein, the term “DMAP” refers to 4-N,N-dimethylamino-pyridine.

As used herein, the term “polar solvent” refers to a solvent having a Polarity Index of above about 2.

The present invention provides a process for preparing zopiclone by combining in a polar organic solvent 6-(5-chloro-2-pyridinyl]-6,7-dihydro-7-hydroxy-5H-pyrrolo[3,4-b]pyrazine-5-one (7-OH-Py) having the formula: 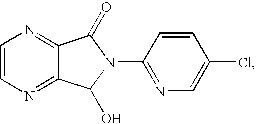
with chloro-carbonyl-4-methyl-piperazine (CMP) having the formula 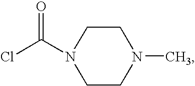
and 4-N,N-dimethylamino-Pyridine (DMAP) and a base. The DMAP acts as a catalyst. The process is conducted in polar solvents and results in zopiclone in high yield and purity without the need of chromatography and use of NaH, typically in yields of 85% to 95%, preferably 90% to 97%, and purities of about 90% to about 99%, preferably 98% to about 99.5%, as measured by area percentage HPLC.

The DMAP can be used in a catalytic amount, avoiding the need for excess of amount of solvents. Further, use of a mild base such an alkyl amine base, instead of NaH, avoids use of anhydrous conditions.

Preferably, the DMAP catalyst is used in a ratio of about 5% to about 50% molar, more preferably about 10% to about 30% molar, and most preferably, in a ratio of about 20% molar in relation to 7-OH-Py.

The base can be a weak organic base or a weak inorganic base. Preferably the organic base is a mono-, di-, or tri-alkylamine wherein the alkyl group may be the same or different and may be C₁₋₆ alkyl, and preferably C₁₋₃ alkyl, preferably methyl or ethyl, and more preferably ethyl. Preferred preferred organic bases are triethylamine or diethylamine.

Preferably, the inorganic base is selected from the group consisting of an alkali metal carbonate and alkali metal bicarbonate. Preferably, the alkali metal carbonate is Na₂CO₃, or K₂CO₃. Preferably, the alkali metal bicarbonate is, NaHCO₃ or/and KHCO₃. Preferably, the base is NaHCO₃.

In one embodiment, the organic base is a C₃-C₁₂ base or C₃-C₉ base preferably triethyl amine or diethyl amine. The inorganic base can be selected from an alkaline carbonate or bicarbonate, preferably Na₂CO₃, K₂CO₃, NaHCO₃ or KHCO₃. Preferably, the carbonate base is NaHCO₃

The amount of the base relative to CMP or its salt is preferably about 2 to about 2.5 moles. The amount of the solvent is preferably about 8-30 ml, preferably about 8-20 and more preferably about 9-12 ml per gram of 7-OH—PY.]

Preferably the solvent is a polar solvent. The most preferred solvents are aprotic polar solvents (ketones, nitriles, esters and chlorinated alkanes).

Specifically, examples of polar solvents include C₃ to C₆ ketones, C₄ to C₆ esters, C₃ to C₈ amides, C₂ to C₄ nitriles and halogenated C₁ to C₆ alkanes. Preferably, the ketone is selected from the group consisting of: methyl-ethyl-ketone, acetone and methyl-iso-butyl-ketone. Preferably, the ester is selected from the group consisting of: ethylacetate and iso-butylacetate. Preferably, the amide is selected from the group consisting of: dimethyl formamide (DMF) and dimethyl acetamide (DMA). Preferably, the nitrile is acetonitrile. Preferably, the halogenated alkane is selected from the group consisting of: methylene chloride and chloroform.

After combining 7-OH-Py, CMP, DMAP and a base in the presence of a polar solvent, a slurry or a solution is obtained. In one embodiment, CMP in a polar solvent is added to a base such as triethyl amine to obtain a slurry followed by addition of DMAP and (7-OH-Py) to the slurry.

Preferably, the slurry or the solution obtained after combining the materials is heated to accelerate the reaction process. Heating is preferably carried out to a temperature of about 40° C. (more preferably about 60° C.) to about the reflux temperature of the solvent. The slurry or the solution can be stirred, such as for about 3 to about 9 hours, more preferably about 3 hours to about 5 hours. After heating, the slurry or the solution can be cooled, preferably to a temperature of about 25° C. to about 0° C., more preferably about 25° C. to about 15° C.

After cooling, the product can be recovered in different ways. In some instances, after cooling, particularly from acetone, the product precipitates in sufficient amount to be recovered without additional work-up. In other instances water is added to the slurry to aid in recovery of the product, such as by causing precipitation of a solid. In other instances addition of water results in a two phase system (an aqueous phase and an organic phase). The product is recovered from the organic phase.

When the product is present as a solid, it can be recovered by conventional techniques such as filtration. The solid can be washed with water, and dried. A suitable drying temperature is about 40° C. to about 80° C. A suitable pressure is below atmospheric pressure, preferably below about 100 mmHg.

When the product is in an organic layer it can be recovered in a similar fashion by removing the solvent, preferably under heating and/or reduced pressure.

In one embodiment the reaction is carried out in MIBK (methyl-iso butyl ketone, MEK (methyl ethyl ketone) or DMF (dimethyl formamide) to obtain a yield of above about 70%, more preferably about 80%, particularly with DMF.

Zopiclone synthesized by the present invention can then be converted to eszopiclone. The conversion of zopiclone to eszopiclone may be done by the method described in the U.S. Pat. No. 6,339,086 or 6,444,673, which process is incorporated herein by reference.

The present invention provides a pharmaceutical composition comprising eszopiclone made by the process of the present invention and at least one pharmaceutically acceptable excipient.

The present invention provides a process for preparing a pharmaceutical formulation comprising combining eszopiclone made by the process of the present invention, with at least one pharmaceutically acceptable excipient.

The present invention provides the use of eszopiclone made by the process of the present invention for the manufacture of a pharmaceutical composition.

The pharmaceutical composition of the present invention can be administered in various dosage forms depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be prepared comprising mixing eszopiclone obtained in the present invention and at least one of other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and die. Some excipients and active ingredients have a tendency to adhere to the surfaces of the punch and die, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the eszopiclone and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxy toluene, butylated hydroxyanisole, and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The solid compositions of the present invention include powders, granulates, aggregates and compacted compositions. The dosage forms include dosage forms suitable for oral, buccal, rectal, parenteral (including subcutaneous, intramuscular, and intravenous), inhalant and ophthalmic administration. Although the most suitable administration route in any given case will depend on the nature and severity of the condition being treated, the most preferred route of the present invention is oral. The dosage forms may be conveniently presented in unit dosage forms and prepared by any of the methods well known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

While the present invention is described with respect to particular examples and preferred embodiments, it is understood that the present invention is not limited to these examples and embodiments. The present invention as claimed therefore includes variations from the particular examples and preferred embodiments described herein, as will be apparent to one of skill in the art.

Having thus described the invention with reference to particular preferred embodiments and illustrative examples, those in the art would appreciate modifications to the invention as describes and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. All references mentioned herein are incorporated in their entirety.

### EXAMPLES

### Example 1

### Preparation of Zopiclone in Ethyl Acetate

To a slurry of 1-chlorocarbonyl-4-methyl piperazine hydrochloride (CMP) (52.96 g) in Ethyl acetate (500 ml), mechanically stirred, was added tri-ethyl amine (Et₃N) (46.12 g) over 10 min. During Et₃N addition, temperature rose by 2° C. After Et₃N addition ended, DMAP (4.62 g) and 6-(5-chloro-2-pyridinyl]-6,7-dihydro-7-hydroxy-5H-pyrrolo[3,4-b]pyrazine-5-one (7-OH-Py) (50 g) were added to the slurry. Then the slurry was heated to 60° C. The slurry was stirred at 60° C. for 7.5 h. The heating was stopped and the slurry was cooled to room temperature. When the temperature reached the room temperature water was added (500 ml) and the slurry was stirred for 1 h. The obtained solid was filtered, washed with acetone (25 ml) and dried in vacuum oven at 40° C. overnight to give zopiclone product crude (71 g yield 90%; purity 99.22%). Zopiclone crude can be purified by crystallization to get zopiclone of a purity greater than 99.8%.

### Example 2

### Preparation of Zopiclone in Iso-Butyl Acetate

To a slurry of 1-chlorocarbonyl-4-methyl piperazine hydrochloride (CMP) (52.96 g) in iso-Butyl acetate (500 ml) were added Et₃N (46.12 g), DMAP (4.62 g) and 7-OH-Py (50 g). The slurry was heated to 80° C. and the heating was maintained for about 5.5 h. After about 2 h at 80° C., 100 ml iso-Butyl acetate was added to the slurry. After the reaction completion (5.5 h, heating), the reaction was stopped and the slurry was cooled to the room temperature. Water (600 ml) was added to the reaction mixture and the slurry was stirred at room temperature for 1 h. The formed solid was filtered, washed with water (50 ml) and acetone (25 ml) and dried in vacuum oven at 40° C. overnight to give zopiclone crude (74.97 g, yield 91.7%, purity 98.86% by HPLC).

### Example 3

### Preparation of Zopiclone in Acetone

1-Chlorocarbonyl-4-methyl piperazine hydrochloride (4.92 g) in acetone (50 ml) was stirred mechanically at room temperature for 5 min. Then Et₃N (4.42 g) was added to the slurry over 10 min. During the tri-ethyl amine (Et₃N) addition, the temperature rose slightly. After Et₃N addition ended, DMAP (0.46 g) was added to the slurry, and after 1-2 min of stirring, 7-OH-Py (5 g) was added. The reaction mixture was heated to reflux and stirred at reflux for 4 h. After 4 h at reflux, heating was stopped and the slurry was cooled to room temperature and ice (˜50 g) was added. Temperature dropped to −6° C. and the slurry was stirred till the temperature reached about 20° C. The solid was filtered, washed with water (10 ml), and dried in vacuum oven at 40° C. overnight to obtain zopiclone crude product (6.95 g yield 89%; purity 99.62% by HPLC).

### Example 4

### Preparation of Zopiclone in CH₂Cl₂

A slurry of 1-chlorocarbonyl-4-methyl piperazine hydrochloride (19.627 g) in CH₂Cl₂ (200 ml) was stirred mechanically and cooled to 5° C. Then tri-ethyl amine (Et₃N) (17.71 g) was added to the slurry over 25 min. During Et₃N addition, there was no temperature rise. After Et₃N addition ended, DMAP (1.85 g) was added to the slurry, and after 1-2 min of stirring, 7-OH-Py (20 g) was added. The slurry changed its appearance at DMAP addition. The reaction mixture was heated to room temperature and stirred for 6 h. After 6 h the stirring was continued at reflux for 1 h. The reaction mixture was cooled to about 20° C. and water was added (70 ml). Phases were separated, with an inter-phase left in organic phase. Organic phase was washed with H₂O (2×70 ml). Inter-phase was filtered. Organic phase was dried over MgSO₄, filtered and the solvent was evaporated to dryness on rotavapor to give zopiclone crude product (28.94 g, yield 95%; purity 98.69% by HPLC).

### Example 5

### Preparation of Zopiclone in N,N-Dimethyl Formamide

A mixture of 1-chlorocarbonyl-4-methyl piperazine hydrochloride (4.92 g) and N,N-dimethyl formamide (DMF) (50 ml,) was stirred mechanically at room temperature. under nitrogen, for 5 min. Then tri-ethyl amine (Et₃N) (4.42 g) was added to slurry over 10 min. During Et₃N addition, the temperature rose by 2° C. After Et₃N addition ended, DMAP (0.46 g) was added to slurry, and after 1-2 min of stirring, 7-OH-Py (5 g) was added. The stirred slurry was heated to 40° C., under nitrogen. The slurry was stirred at 40° C. for 5 h under nitrogen After 5 h at 40° C., the slurry was cooled to room temperature and stirred overnight (˜15 h.). After that, the solid was filtered and discarded; 50 ml ice-cold water were added to the filtrate. A solid precipitated and the slurry was stirred 1 h at this temperature, then the solid was filtered, washed with water (20 ml) and di-isopropyl ether (20 ml), then dried in vacuum oven at 40° C. to give zopiclone crude product (6.34 g, yield 79.1%; purity 97% by HPLC).

### Example 6

### Preparation of Zopiclone in ACN

To a slurry of 1-chlorocarbonyl-4-methyl piperazine hydrochloride (CMP) (4.92 g) in acetonitrile (ACN) (75 ml), stirred mechanically at Room temperature and under nitrogen, was added tri-ethyl amine (Et₃N) (4.42 g). After Et₃N addition ended, DMAP (0.46 g) was added to the slurry, and after 1-2 min of stirring, 7-OH-Py (5 g) was added. The slurry changed its appearance at 7-OH addition. The stirring was applied for 2 h at about 0° C., then at room temperature for 14 h. The reaction was completed after an additional heating (about 9 h) at 40° C. After cooling to the room temperature the solvent was evaporated, to give a yellowish-brown solid, that was dissolved in CH₂Cl₂ (40 ml) and water (50 ml). Phases were separated while the interphase was left in the aqueous phase. The aqueous phase was extracted with CH₂Cl₂ (40 ml). The combined organic phases were washed with water (50 ml), dried over MgSO₄ filtered and evaporated to dryness to give zopiclone crude product (6.17 g, yield 82.2%; purity 98.24% by HPLC).

### Example 7

### Preparation of Zopiclone in Acetonitrile

To a slurry of 1-chlorocarbonyl-4-methyl piperazine hydrochloride (CMP) (5.67 g) in acetonitrile (ACN) (150 ml), mechanically stirred under nitrogen at 0° C., were added Na₂CO₃ (4.98 g) and DMAP (0.46 g) followed by addition of 7-OH-Py (5 g). The reaction mixture was then stirred at 2° C. for 1.5 h, under nitrogen followed by 18 h stirring at room temperature and a few hours at reflux. After the reaction completion, the solvent was evaporated, to give a solid that was re-dissolved in Ethyl acetate and water. Phases were separated and the aqueous phase was extracted with Ethyl acetate. The solid formed in the aqueous layer was filtered to give zopiclone crude product (6.13 g, yield 64.9%; purity 99.85% by HPLC).

Other examples are summarized in the following table:

| | | | | | Purity profile, HPLC % area | | | |
|---|---|---|---|---|---|---|---|---|
| Exp. | Solvent | Base | Temp. | Yield | rrt: 0.68 | 7-OH-Py | Zopiclone | rrt: 2.3 |
| AM-615 | MIBK | Et₃N | 60° C. | 75% | 0.03 | 0.86 | 99.11 | |
| AM-617 | MEK | Et₃N | 60° C. | 72.97% | 0.03 | 0.21 | 99.76 | |
| AM-603 | CAN | K₂CO₃ | 81° C. | 17.60% | 0.03 | 0.04 | 99.84 | |
| AM-606 | DMF | Na₂CO₃ | 40° C. | 82.50% | 0.02 | 0.02 | 99.88 | 0.05 |

### Example 8

### Preparation of Eszopiclone starting from Zopiclone

a) Preparation of Eszopiclone D-(+)-Malate

To the mixture of zopiclone prepared according to the example 2 (15 g) in method (152 ml) and acetone (283 ml) was added D-(+)-malic acid (5.27 g) and the mixture was heated to 48° C. Complete dissolution was obtained. After seeding with D-(+)-eszopiclone malate, the reaction mixture was cooled over four hours to about 10° C. obtained solid was filtrated and washed with methanol. After drying in vacuum oven at 40° C. the product eszopiclone D-(+)-malate was obtained (8.73 g; yield 88.0%; optical purity 98.2% eszopiclone).

b) Preparation of Eszopiclone Crude

Eszopiclone D-(+)-malate (8.47 g) was dissolved in water (212 ml) at room temperature. The solution was treated with active carbon for color improvement. Sodium carbonate (1.94 g) was added in portion. The solid obtained was filtrated, and washed with water. The obtained wet material (6.29 g; LOD 17.8%; yield 82.1%;) is eszopiclone free base crude and was used for the next step without drying.

c) Preparation of Eszopiclone Cryst

Wet eszopiclone crude (5.28 g) was crystallized from aqueous iso-propanol (10% water) (85 ml); complete dissolution was obtained at about 80° C. The solid obtained on cooling was filtrated at 10° C., washed with iso-propanol and dried in vacuum-oven at 40° C. The dried material is eszopiclone cryst. (3.83 g; yield 88.2%; optical purity 99.98%; chemical purity 99.97% by HPLC . . .

1. A process for preparing zopiclone comprising combining in a polar solvent 6-(5-chloro-2-pyridinyl]-6,7-dihydro-7-hydroxy-5H-pyrrolo[3,4-b]pyrazine-5-one (7-OH-Py) having the formula: 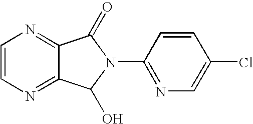
with chloro-carbonyl-4-methyl-piperazine (CMP) free base or as an acid addition salt having the formula: 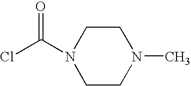
and 4-N,N-dimethylamino-Pyridine (DMAP) and a base to obtain zopiclone.

2. The process of claim 1, wherein the concentration is about 5% to about 50% molar DMAP catalyst in relation to 7-OH-Py.

3. The process of claim 2, wherein the ratio is of about 10% to about 30%.

4. The process of claim 3, wherein the ratio is of about 20%.

5. The process of claim 1, wherein the base is an organic base.

6. The process of claim 5, wherein the organic base is a C₃-C₁₂ base.

7. The process of claim 5, wherein the organic base is a C₃-C₉ base.

8. The process of claim 7, wherein the organic base is triethyl amine or diethyl amine.

9. The process of claim 8, wherein the base is inorganic.

10. The process of claim 8, wherein the inorganic base is an alkaline carbonate or bicarbonate.

11. The process of claim 9, wherein the base is Na₂CO₃, K₂CO₃, NaHCO₃ or KHCO₃.

12. The process of claim 10, wherein the carbonate base is NaHCO₃.

13. The process of claim 1, wherein the polar solvent is a C₃ to C₆ ketones, C₄ to C₈ esters, C₃ to C₆ amides, nitriles or halogenated C₁ to C₆ alkanes.

14. The process of claim 13, wherein the ketone is selected from the group consisting of: methyl-ethyl-ketone, acetone and methyl-iso-butyl-ketone.

15. The process of claim 13, wherein the ester is selected from the group consisting of: ethylacetate and iso-butylacetate.

16. The process of claim 13, wherein the amide is selected from the group consisting of: dimethyl formamide (DMF) and dimethyl acetamide (DMA).

17. The process of claim 13, wherein the nitrile is acetonitrile.

18. The process of claim 13, wherein the halogenated alkane is selected from the group consisting of: methylene chloride and chloroform.

19. The process of claim 18, wherein after combining, a slurry or a solution is obtained.

20. The process of claim 19, wherein CMP in a polar solvent is added to a base to obtain a slurry followed by addition of DMAP and (7-OH-Py) to the slurry.

21. The process of claim 1, further comprising a step of heating after combining.

22. The process of claim 1, wherein the heating is carried out of a temperature of about 60° C. to about the reflux temperature of the solvent.

23. The process of claim 1, further comprising a step of cooling after heating.

24. The process of claim 23, wherein cooling is carried out at a temperature of about 25° C. to about 0° C.

25. The process of claim 23, further comprising recovery of the zopiclone after cooling.

26. The process of claim 25, wherein water is added to the slurry to aid in recovery of the zopiclone.

27. The process of claim 26, wherein addition of water results in a two phase system, having an aqueous phase and an organic phase, wherein zopiclone moves to the organic phase.

28. The process of claim 25, further comprising a step of drying the recovered zopiclone.

29. The process of claim 28, wherein drying is carried out at a temperature of about 40° C. to about 80° C.

30. The process of claim 28, wherein drying is carried out at below about atmospheric pressure.

31. The process of claim 30, wherein the pressure is below about 100 mmHg.

32. The process of claim 1, wherein CMP is used as free base.

33. The process of claim 1, wherein the acid addition salt of CMP is HCl.

34. A process for preparing eszopiclone comprising converting zopiclone of claim 1 to eszopiclone.

35. A pharmaceutical composition comprising eszopiclone of claim 34 and at least one pharmaceutically acceptable excipient.
